(19) United States
(12) Patent Application Publication
(10) Pub. No.: US 2026/0346047 A1
(43) Pub. Date: Nov. 5, 2026

(54) Capsule Feeding Assembly

(71) Applicant: Mingao Medical Technology (Kunshan) Co., Ltd., Kunshan City (CN)

(72) Inventors: Wenpin WANG, Kunshan City (CN); Jingyang LU, Kunshan City (CN)

(21) Appl. No.: 19/433,672

(22) Filed: Dec. 26, 2025

(30) Foreign Application Priority Data
Apr. 30, 2025 (CN) 2025208604944

(51) Int. Cl. A61N 5/10; A61J 1/03; A61J 1/14

(52) U.S. Cl. CPC A61N 5/1007; A61J 1/03; A61J 1/1418; A61J 1/1468; A61N 2005/1094

(57) ABSTRACT

A capsule feeding assembly includes a radiation-proof container, a capsule tube and a radiation-proof cover. The radiation-proof container is provided with a containing groove and a guide groove, the guide groove is provided on an upper side of the containing groove and communicates with the containing groove; the guide groove is provided obliquely toward the containing groove; a capsule tube is contained in the containing groove and can enter or exit the containing groove under a guidance of the guide groove; the capsule tube is provided with an inner cavity, which is used for storing radioactive capsule drugs, and the capsule tube provides external protection for the radioactive capsule drugs; the radiation-proof cover is detachably connected to the radiation-proof container and can cover or open the containing groove and the guide groove; the radiation-proof cover is provided with an operation portion.

### CROSS-REFERENCE TO RELATED APPLICATIONS

This application claims priority to Chinese Patent Application No. 202520860494.4, filed on Apr. 30, 2025, the entire disclosure of which is incorporated herein by reference.

### TECHNICAL FIELD

The present application relates to the technical field of a capsule feeding assembly, and relates to a capsule feeding assembly.

### BACKGROUND

With the development of science and technology, radioactive capsule drugs are widely and deeply applied in the field of biology. The core lies in the combination of radionuclides and specific biomolecules, thereby realizing the tracking of in vivo biological processes, disease diagnosis or treatment. The capsule feeding assembly is used to store radioactive capsule drugs.

In the existing technology, the existing capsule feeding assembly includes a container and a cover. The container is used for storing radioactive capsule drugs, and the cover is detachably connected to the container. However, the radioactive capsule drugs are in a disorderly state in the container, which is inconvenient for subsequent feeding, resulting in poor usability of the existing capsule feeding assembly.

### SUMMARY

The purpose of the present application is to provide a capsule feeding assembly. A radiation-proof container is provided with a containing groove and a guide groove, the guide groove is provided on an upper side of the containing groove and communicates with the containing groove; the guide groove is provided obliquely toward the containing groove; a capsule tube is contained in the containing groove and can enter or exit the containing groove under a guidance of the guide groove; a capsule tube is provided with an inner cavity, which is used for storing radioactive capsule drugs, and the capsule tube provides external protection for the radioactive capsule drugs; a radiation-proof cover is detachably connected to the radiation-proof container and can cover or open the containing groove and the guide groove; the radiation-proof cover is provided with an operation portion, which is provided on an outer side wall of the radiation-proof cover and exposed to the external environment, so that the radioactive capsule drugs can be orderly placed in the containing groove through the capsule tube, facilitating subsequent feeding. Meanwhile, the capsule tube can protect the radioactive capsule drugs and has a radiation-proof effect, improving the usability of the capsule feeding assembly.

To achieve the above purpose, the present application provides the following technical solutions: a capsule feeding assembly, including:
a radiation-proof container, provided with a containing groove and a guide groove, wherein the guide groove is provided on an upper side of the containing groove and communicates with the containing groove, and the guide groove is obliquely provided toward the containing groove;
a capsule tube, contained in the containing groove and capable of entering or exiting the containing groove under a guidance of the guide groove; wherein the capsule tube is provided with an inner cavity for storing radioactive capsule drugs, and the capsule tube is configured to provide external protection for the radioactive capsule drugs; and
a radiation-proof cover, detachably connected to the radiation-proof container and capable of covering or opening the containing groove and the guide groove; wherein the radiation-proof cover is provided with an operation portion, and the operation portion is provided on an outer side wall of the radiation-proof cover and exposed to external environment.

### BRIEF DESCRIPTION OF THE DRAWINGS

To more clearly illustrate the technical solutions in the embodiments of the present application, the accompanying drawings needed in the description of the embodiments will be briefly introduced below. Obviously, the drawings in the following description are only some embodiments of the present application, and those skilled in the art can obtain other drawings based on these drawings without creative work. For a more complete understanding of the present application and its beneficial effects, the following description will be made in conjunction with the accompanying drawings. In the following description, the same reference numerals denote the same parts.

FIG. 1 shows a schematic diagram of a capsule feeding assembly according to an embodiment of the present application.

FIG. 2 shows a cross-sectional view of a capsule feeding assembly according to an embodiment of the present application.

FIG. 3 shows an exploded view of a capsule feeding assembly according to an embodiment of the present application.

FIG. 4 shows a cross-sectional view of FIG. 3.

FIG. 5 shows a cross-sectional view of a feeding tube of the capsule feeding assembly inserted into the radiation-proof container according to an embodiment of the present application.

FIG. 6 shows a cross-sectional view of a connection between the feeding tube and the capsule tube of the capsule feeding assembly according to an embodiment of the present application.

FIG. 7 shows a cross-sectional view of the entire feeding tube of the capsule feeding assembly taken out of the radiation-proof container according to an embodiment of the present application.

FIG. 8 shows a cross-sectional view of the feeding tube and the capsule tube of the capsule feeding assembly according to an embodiment of the present application.

FIG. 9 shows a cross-sectional view of a opening-closing cover of the capsule feeding assembly being detached from the feeding tube according to an embodiment of the present application.

### DETAILED DESCRIPTION OF THE EMBODIMENTS

The following will clearly and completely describe the technical solutions in the embodiments of the present application in conjunction with the accompanying drawings in the embodiments of the present application. Obviously, the described embodiments are only a part of the embodiments of the present application, not all of them. Based on the embodiments in this application, all other embodiments obtained by those skilled in the art without creative work shall fall within the protection scope of this application.

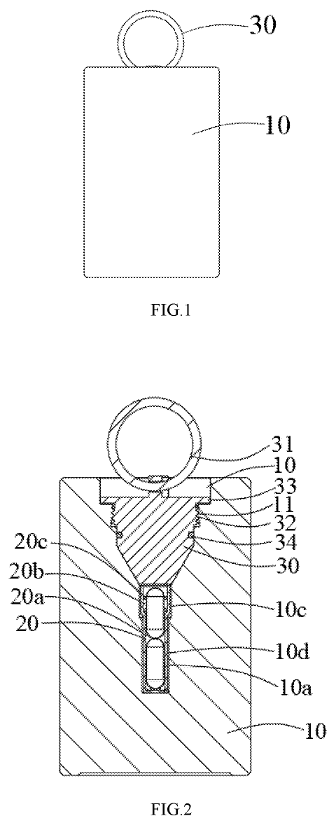

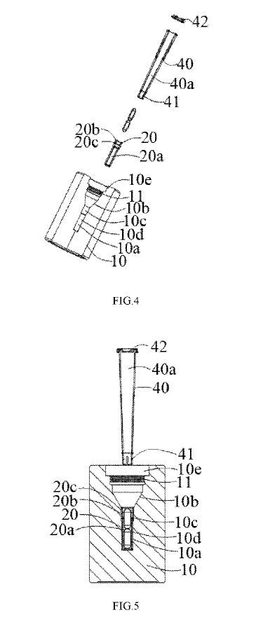

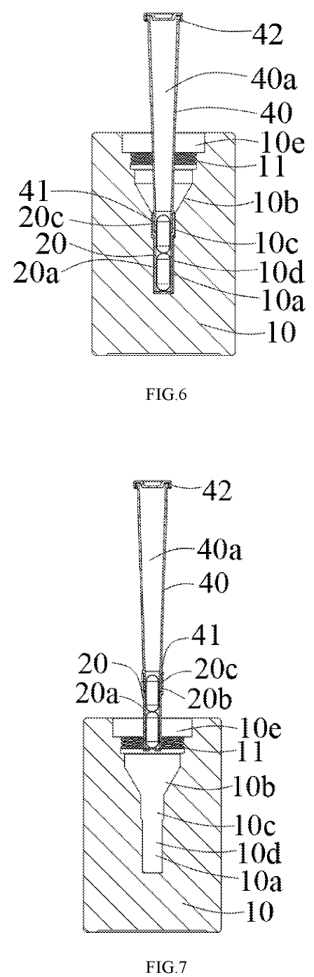

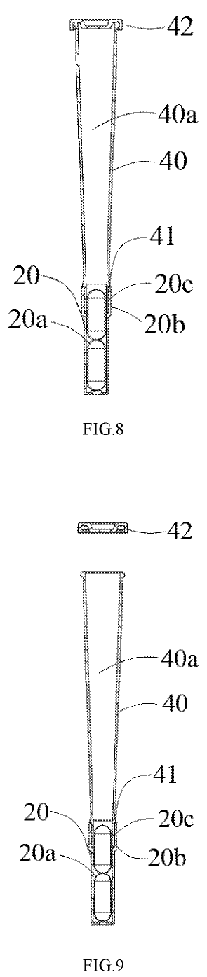

Please refer to FIGS. 1-9. The embodiment of the present application provides a capsule feeding assembly 100, which is used to store radioactive capsule drugs.

Please refer to FIGS. 1-9. In the embodiment of the present application, the capsule feeding assembly 100 includes a radiation-proof container 10, a capsule tube 20 and a radiation-proof cover 30. The radiation-proof container 10 is provided with a containing groove 10a and a guide groove 10b. The guide groove 10b is provided on an upper side of the containing groove 10a and communicates with the containing groove 10a; the guide groove 10b is provided obliquely toward the containing groove 10a; the capsule tube 20 is contained in the containing groove 10a and can enter or exit the containing groove 10a under a guidance of the guide groove 10b; the capsule tube 20 is provided with an inner cavity 20a, which is used for storing radioactive capsule drugs, and the capsule tube 20 provides external protection for the radioactive capsule drugs; the radiation-proof cover 30 is detachably connected to the radiation-proof container 10 and can cover or open the containing groove 10a and the guide groove 10b; the radiation-proof cover 30 is provided with an operation portion 31, which is provided on an outer side wall of the radiation-proof cover 30 and exposed to the external environment, so that the radioactive capsule drugs can be orderly placed in the containing groove 10a through the capsule tube 20, facilitating subsequent feeding. And the capsule tube 20 can protect the radioactive capsule drugs and has a radiation-proof effect, improving the usability of the capsule feeding assembly 100.

Please refer to FIGS. 1-9. In the embodiment of the present application, the radiation-proof container 10 is provided with a containing groove 10a and a guide groove 10b. The containing groove 10aserves as an internal space of the radiation-proof container 10. The guide groove 10bis provided on the upper side of the containing groove 10a and communicates with the containing groove 10a; the guide groove 10b is provided obliquely toward the containing groove 10a.

The capsule tube 20 is contained in the containing groove 10a and can enter or exit the containing groove 10a under a guidance of the guide groove 10b, which improves the entering smoothness of the capsule tube 20 and ensures the position accuracy of the capsule tube 20 relative to the radiation-proof container 10. The capsule tube 20 is provided with an inner cavity 20a, which is used for storing radioactive capsule drugs, and the capsule tube 20 provides external protection for the radioactive capsule drugs. In this way, the radioactive capsule drugs can be orderly placed in the containing groove 10a through the capsule tube 20, facilitating subsequent feeding. And the capsule tube 20 can protect the radioactive capsule drugs and has a radiation-proof effect, improving the usability of the capsule feeding assembly 100.

The radiation-proof cover 30 is provided on the upper side of the radiation-proof container 10, and the radiation-proof cover 30 is detachably connected to the radiation-proof container 10, so that the radiation-proof cover 30 can be connected to or separated from the radiation-proof container 10, and the radiation-proof cover 30 can cover or open the containing groove 10a and the guide groove 10b; when the radiation-proof cover 30 covers the containing groove 10a and the guide groove 10b, it is convenient for the radiation-proof cover 30 to block the radioactive capsule drugs in the capsule tube 20, avoiding the radioactive capsule drugs from accidentally separating from the capsule tube 20. When the radiation-proof cover 30 opens the containing groove 10a and the guide groove 10b, it is convenient for the capsule tube 20 to enter or exit the containing groove 10a, improving the convenience of taking and placing the radioactive capsule drugs in the capsule tube 20.

The radiation-proof cover 30 is provided with an operation portion 31, which is provided on an outer side wall of the radiation-proof cover 30 and exposed to the external environment, so that the user can operate the radiation-proof cover 30 to connect or separate from the radiation-proof container 10 through the operation portion 31.

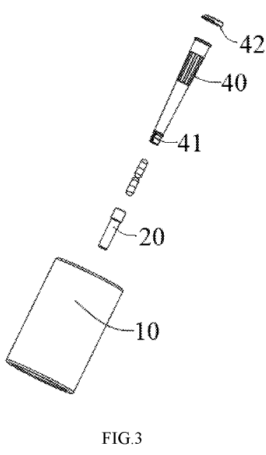

Please refer to FIGS. 1-2. In the embodiment of the present application, the radiation-proof container 10 is provided with a first connection portion 11, and the first connection portion 11, the guide groove 10b and the containing groove 10a are provided in an up-down direction; so that the first connection portion 11, the guide groove 10b and the containing groove 10a can make full use of the internal space of the radiation-proof container 10. The guide groove 10b is an annular groove and converges to the containing groove 10a along an oblique direction; the guide groove 10b and the containing groove 10a are provided in a step manner. The step arrangement increases the sense of hierarchy and complexity of the structure, which can disperse stress when subjected to external impact, improve the overall deformation resistance of the radiation-proof container 10, and better protect the radioactive capsule drugs in the capsule tube 20.

Please refer to FIGS. 1-2. In the embodiment of the present application, the containing groove 10a is provided with a first sub-containing groove 10c and a second sub-containing groove 10d. The first sub-containing groove 10c is located on an upper side of the second sub-containing groove 10d and communicates with the second sub-containing groove 10d and the guide groove 10b; so that the guide groove 10b, the first sub-containing groove 10c and the second sub-containing groove 10d are provided sequentially from top to bottom, thus facilitating the capsule tube 20 to enter the second sub-containing groove 10d through the guide groove 10b and the first sub-containing groove 10c in sequence. A groove diameter of the first sub-containing groove 10c is larger than that of the second sub-containing groove 10d, so that the first sub-containing groove 10c and the second sub-containing groove 10d are provided in a step manner, and the second sub-containing groove 10d is adapted to the capsule tube 20, so that an inner side wall of the second sub-containing groove 10d can limit the position of the capsule tube 20, thereby preventing the capsule tube 20 from shaking relative to the second sub-containing groove 10d.

Please refer to FIGS. 1-2. In the embodiment of the present application, the first connection portion 11 is a threaded portion, and the radiation-proof cover 30 is provided with a second connection portion 32. The radiation-proof cover 30 rotates relative to the radiation-proof container 10, and the second connection portion 32 is screwed to the first connection portion 11 along a rotation direction, so that the radiation-proof cover 30 can be threadedly connected to the radiation-proof container 10 through the cooperation of the second connection portion 32 and the first connection portion 11, so as to realize the detachable connection of the radiation-proof cover 30 relative to the radiation-proof container 10. When the radiation-proof cover 30 rotates clockwise relative to the radiation-proof container 10, the radiation-proof cover 30 is connected to the radiation-proof container 10. When the radiation-proof cover 30 rotates counterclockwise relative to the radiation-proof container 10, the radiation-proof cover 30 is separated from the radiation-proof container 10.

Please refer to FIGS. 1-2. In the embodiment of the present application, the radiation-proof container 10 is further provided with a first step groove 10e, and the first step groove 10e is provided on a side of the first connection portion 11 opposite to the guide groove 10b; the radiation-proof cover 30 is provided with a first step portion 33, the first step portion 33 is adapted to the first step groove 10e, and the first step portion 33 is provided on an upper side of the second connection portion 32; both the first step portion 33 and the first step groove 10e are provided in an annular shape, and the first step portion 33 gradually approaches the first step groove 10e as the second connection portion 32 rotates relative to the first connection portion 11, so that the radiation-proof cover 30 and the radiation-proof container 10 can be accurately docked, thereby enhancing the sealing performance between the radiation-proof cover 30 and the radiation-proof container 10.

Please refer to FIGS. 1-2. In the embodiment of the present application, the radiation-proof cover 30 is sleeved with a sealing ring 34, and the sealing ring 34 is located between the radiation-proof cover 30 and the radiation-proof container 10 and hermetically connects the radiation-proof cover 30 and the radiation-proof container 10, so that the radiation-proof cover 30 and the radiation-proof container 10 can compress the sealing ring 34, further enhancing the sealing performance between the radiation-proof cover 30 and the radiation-proof container 10 and preventing external gas or liquid from entering the radiation-proof container 10.

Please refer to FIGS. 1-9. In the embodiment of the present application, the capsule feeding assembly 100 further includes a feeding tube 40; the feeding tube 40 is provided with a through hole 40a; when the radiation-proof container 10 is in an open state, the feeding tube 40 penetrates through the guide groove 10b and enters the containing groove 10a. At this time, the feeding tube 40 is inserted into the capsule tube 20 and is in a connected state with the capsule tube 20, and the through hole 40a communicates with the inner cavity 20a of the capsule tube 20 and exposes the radioactive capsule drugs, so that the feeding tube 40 can drive the capsule tube 20 to separate from the containing groove 10a, improving the convenience of taking and placing the radioactive capsule drug in the capsule tube 20.

Please refer to FIGS. 1-9. In the embodiment of the present application, the capsule tube 20 is provided with a second step groove 20b, and an inner side wall of the second step groove 20b is provided with a first snapping portion 20c; an outer side wall of an end of the feeding tube 40 is provided with a second snapping portion 41, and the second snapping portion 41 and the first snapping portion 20c form a snapping connection structure, so that the feeding tube 40 can be connected to the capsule tube 20 through the cooperation of the second snapping portion 41 and the first snapping portion 20c, thereby facilitating the feeding tube 40 to drive the capsule tube 20 to separate from the containing groove 10a in the external environment.

Please refer to FIGS. 1-9. In the embodiment of the present application, the through hole 40a is provided in a conical shape, and a minimum aperture of the through hole 40a is larger than an outer diameter of the radioactive capsule drug, so that the radioactive capsule drug can enter the capsule tube 20 along an inner side wall of the through hole 40a.

Please refer to FIGS. 1-9. In the embodiment of the present application, when the feeding tube 40 is separated from the radiation-proof container 10, the capsule tube 20 is gradually separated from the radiation-proof container 10 along with the separation of the feeding tube 40. At this time, the capsule tube 20 is in a connected state with the feeding tube 40, and the inner cavity 20a of the capsule tube 20 is connected to the through hole 40a, so that the radioactive capsule drug in the capsule tube 20 can flow to the external environment through the through hole 40a.

Please refer to FIGS. 1-9. The feeding tube 40 is sleeved with an opening-closing cover 42, and the opening-closing cover 42 is detachably connected to the feeding tube 40, so that the opening-closing cover 42 can be connected to or separated from the feeding tube 40, and the opening-closing cover 42 can open or close a discharge port of the through hole 40a; the radioactive capsule drug is discharged outward from the inner cavity 20a of the capsule tube 20 and the discharge port of the through hole 40a, so as to feed the radioactive capsule drug to the user.

Compared with the related art, the beneficial effects of the present application are:
the present application provides a capsule feeding assembly 100. A radiation-proof container 10 is provided with a containing groove 10a and a guide groove 10b, the guide groove 10b is provided on an upper side of the containing groove 10a and communicates with the containing groove 10a; the guide groove 10b is provided obliquely toward the containing groove 10a; a capsule tube 20 is contained in the containing groove 10a and can enter or exit the containing groove 10a under a guidance of the guide groove 10b; the capsule tube 20 is provided with an inner cavity 20a, which is used for storing radioactive capsule drugs, and the capsule tube 20 provides external protection for the radioactive capsule drugs; a radiation-proof cover 30 is detachably connected to the radiation-proof container 10 and can cover or open the containing groove 10a and the guide groove 10b; the radiation-proof cover 30 is provided with an operation portion 31, which is provided on an outer side wall of the radiation-proof cover 30 and exposed to the external environment, so that the radioactive capsule drugs can be orderly placed in the containing groove 10a through the capsule tube 20, facilitating subsequent feeding. Meanwhile, the capsule tube 20 can protect the radioactive capsule drugs and has a radiation-proof effect, improving the usability of the capsule feeding assembly 100.

It should be noted that all directional indications (such as up, down, left, right, front, back, etc.) in the embodiments of the present application are only used to explain the relative positional relationship and movement between components in a specific posture. If the specific posture changes, the directional indication will also change accordingly.

It should also be noted that when an element is referred to as being "fixed on" or "provided on" another element, it can be directly on the other element or there may be an intermediate element at the same time. When an element is referred to as being "connected" to another element, it can be directly connected to the other element or there may be an intermediate element at the same time.

In addition, the descriptions involving "first", "second", etc. in the present application are only for descriptive purposes, and cannot be understood as indicating or implying their relative importance or implicitly indicating the number of indicated technical features. Therefore, the features defined with "first" and "second" may explicitly or implicitly include at least one such feature. In addition, the technical solutions between the various embodiments can be combined with each other, but must be based on the realization by those skilled in the art. When the combination of technical solutions is contradictory or cannot be realized, it should be considered that such a combination of technical solutions does not exist and is not within the protection scope required by the present application.

The above descriptions are only preferred embodiments of the present application, and are not intended to limit the patent scope of the present application. All equivalent structural transformations made under the inventive concept of the present application by using the contents of the description and FIGS., or directly/indirectly applied in other related technical fields, are included in the patent protection scope of the present application.

What is claimed is:

1. A capsule feeding assembly, comprising:
a radiation-proof container, provided with a containing groove and a guide groove, wherein the guide groove is provided on an upper side of the containing groove and communicates with the containing groove, and the guide groove is obliquely provided toward the containing groove;
a capsule tube, contained in the containing groove and capable of entering or exiting the containing groove under a guidance of the guide groove; wherein the capsule tube is provided with an inner cavity for storing radioactive capsule drugs, and the capsule tube is configured to provide external protection for the radioactive capsule drugs; and
a radiation-proof cover, detachably connected to the radiation-proof container and capable of covering or opening the containing groove and the guide groove; wherein the radiation-proof cover is provided with an operation portion, and the operation portion is provided on an outer side wall of the radiation-proof cover and exposed to external environment.

2. The capsule feeding assembly according to claim 1, wherein the radiation-proof container is provided with a first connection portion, and the first connection portion, the guide groove and the containing groove are provided in an up-down direction;
the guide groove is an annular groove and converges to the containing groove along an oblique direction; and
the guide groove and the containing groove are provided in a step manner.

3. The capsule feeding assembly according to claim 2, wherein the containing groove is provided with a first sub-containing groove and a second sub-containing groove, the first sub-containing groove is located on an upper side of the second sub-containing groove and communicates with the second sub-containing groove and the guide groove;
a groove diameter of the first sub-containing groove is larger than that of the second sub-containing groove; and
the second sub-containing groove is adapted to the capsule tube.

4. The capsule feeding assembly according to claim 2, wherein the first connection portion is a threaded portion, the radiation-proof cover is provided with a second connection portion, the radiation-proof cover rotates relative to the radiation-proof container, and the second connection portion is screwed to the first connection portion along a rotation direction.

5. The capsule feeding assembly according to claim 4, wherein the radiation-proof container is further provided with a first step groove, and the first step groove is provided on a side of the first connection portion opposite to the guide groove;
the radiation-proof cover is provided with a first step portion, the first step portion is adapted to the first step groove, and the first step portion is provided on a side of the second connection portion; and
the first step portion and the first step groove are provided in an annular shape, and the first step portion gradually approaches the first step groove as the second connection portion rotates relative to the first connection portion.

6. The capsule feeding assembly according to claim 4, wherein the radiation-proof cover is sleeved with a sealing ring, and the sealing ring is located between the radiation-proof cover and the radiation-proof container and hermetically connects the radiation-proof cover and the radiation-proof container.

7. The capsule feeding assembly according to claim 1, wherein the capsule feeding assembly further comprises a feeding tube;
the feeding tube is provided with a through hole;
when the radiation-proof container is in an open state, the feeding tube penetrates through the guide groove and enters the containing groove, and the feeding tube is inserted into the capsule tube and is in a connected state with the capsule tube, and
the through hole communicates with the inner cavity of the capsule tube and exposes the radioactive capsule drugs.

8. The capsule feeding assembly according to claim 7, wherein the capsule tube is provided with a second step groove, and an inner side wall of the second step groove is provided with a first snapping portion;
an outer side wall of an end of the feeding tube is provided with a second snapping portion, and the second snapping portion and the first snapping portion form a snapping connection structure.

9. The capsule feeding assembly according to claim 8, wherein the through hole is provided in a conical shape, and a minimum aperture of the through hole is larger than an outer diameter of each radioactive capsule drug.

10. The capsule feeding assembly according to claim 7, wherein when the feeding tube is separated from the radiation-proof container, the capsule tube is gradually separated from the radiation-proof container along with a separation of the feeding tube, and the capsule tube is in a connected state with the feeding tube, and the inner cavity of the capsule tube is communicated with the through hole;
the feeding tube is sleeved with an opening-closing cover, and the opening-closing cover is detachably connected to the feeding tube and opens or closes a discharge port of the through hole; and
each radioactive capsule drug is discharged outward from the inner cavity of the capsule tube and the discharge port of the through hole.
